(19) United States
(12) Patent Application Publication
(10) Pub. No.: US 2015/0313829 A1
(43) Pub. Date: Nov. 5, 2015

(54) TOPICAL FORMULATIONS FOR THE PREVENTION AND TREATMENT OF ALOPECIA AND INHIBITION OF HAIR GROWTH

(71) Applicants: Roberto QUEIROGA LAUTNER, (US); Daniel CAMPOS VILLELA, (US); Rodrigo ARAUJO FRAGA DA SILVA, (US); Anderson José FERREIRA, (US); Elisangela Fátima DA SILVA, (US); UNIVERSIDADE FEDERAL DE MINAS GERAIS -UFMG, Belo Horizonte (BR)

(72) Inventors: Frederic Jean Georges Frezard, Belo Horizonte (BR); Robson Augusto Souza Dos Santos, Belo Horizonte (BR); Ana Paula Correa De Oliveira Bahia, Belo Horizonte (BR); Neiva Caldeira Silva, Belo Horizonte (BR); Roberto Queiroga Lautner, Belo Horizonte (BR); Daniel Campos Villela, Belo Horizonte (BR); Rodrigo Araujo Fraga Da Silva, Nova Lima (BR); Anderson Jose Ferreira, Belo Horizonte (BR); Elisangela Fatima Da Silva, Belo Horizonte (BR)

(21) Appl. No.: 14/647,348

(22) PCT Filed: Nov. 26, 2013
(86) PCT No.: PCT/BR2013/000558

§ 371 Date: May 26, 2015

(30) Foreign Application Priority Data
Nov. 26, 2012 (BR) BR1020120300680
Nov. 25, 2013 (BR) BR1020130301515

(51) Int. Cl. A61K 8/64; A61K 8/55; A61Q 7/02; A61Q 7/00

(52) U.S. Cl. CPC A61K 8/64; A61Q 7/00; A61K 8/553; A61Q 7/02

(57) ABSTRACT

The present invention discloses formulations for the treatment and prevention of alopecia; the formulations comprise angiotensin-(1-7) and/or their analogues, encapsulated in ultradeformable vesicles prepared from specific combinations of phospholipids and surfactants, which confer efficacy to topical application in the control of hair growth, which indicates their great potential for the prevention and treatment of alopecia. The invention also describes formulations containing A779 Mas receptor antagonist of angiotensin-(1-7) and/or their analogues and their use in inhibiting hair growth.

### CROSS-REFERENCE TO RELATED APPLICATIONS

This application is a 371 national stage application of PCT Application No. PCT/BR2013/000558, filed Nov. 26, 2013, and claims the benefit of Brazilian Application No. BR1020130301515, filed on Nov. 25, 2013, and Brazilian Application No. BR1020120300680, filed Nov. 26, 2012; the entire contents of which are hereby incorporated by reference as if fully set forth herein.

### BACKGROUND

The present invention discloses formulations for the treatment and prevention of alopecia; the formulations comprise angiotensin-(1-7) and/or their analogues, encapsulated in ultradeformable vesicles prepared with specific combinations of phospholipids and surfactants, which confer efficacy in topical application in the control of hair growth, which indicates their great potential for the prevention and treatment of alopecia. The invention also describes formulations containing A-779, a Mas receptor antagonist of angiotensin (1-7) and/or their analogues and their use in inhibiting hair growth.

### SUMMARY

Androgenetic alopecia or baldness is a physiological event that occurs in genetically predisposed individuals, leading to “hair loss.” Androgenetic alopecia occurs by accelerating the growth cycle. The hair follicles produce thinner, shorter and depigmented hair, in a miniaturization process sometimes referred to as “wire.” There is an increase in the activity of 5-α-reductase enzyme in men and specific dehydrogenase enzymes within the hair follicle in women. This enzyme is responsible for converting testosterone to dihydrotestosterone, which has high affinity to androgen receptors in dermal papilla (Wiedemeyer K., Schill WB, Loser C. Diseases on Hair Follicles Hair Loss Leading to Part I: . . . Nonscarring alopecia Dermatology for the Clinician, JulyAugust, p. 209-214, 2004). Although the genes have not yet been identified, it is known that there is a genetic predisposition to the onset of alopecia, which is present in varying degrees, locations and ages.

The non-surgical treatment of androgenetic alopecia consists mainly of three drugs: finasteride (Propecia®), a selective inhibitor of 5-α-reductase enzyme administered orally, 17-α-estradiol (Avicis®) and minoxidil (Rogaine®) administered topically (Wiedemeyer et al., 2004).

Such treatments have variable effectiveness among individuals. Not all respond in the same manner to the above mentioned treatments, moreover, the interruption of the treatment leads to the return of the hair loss. Other frequently reported limitations of such treatments are the appearance of local irritations such as dry skin or itching and scaling of the scalp after chronic application of minoxidil or lotions of α-estradiol and the decrease of libido and erectile dysfunction, in the case of finasteride (Bull the medicine Avicis®, available in http://www.medicinanet.com.br/bula/744/avicis.htm, accessed Nov. 19, 2012; Bull of medicine Propecia®, available at http: //www.medicinanet. com.br/bula/4314/propecia.htm, accessed Nov. 19, 2012; Bull of Rogaine® drug, available in http://www.medicinanet.com.br/bula/4444/regaine_{—}2.htm, accessed Nov. 19, 2012). In this context, it is important to develop new bioactive agents and new cosmetic formulations/drugs that can be used to prevent, delay or treat androgenetic alopecia. In 2000, the effect of endogenous peptide angiotensin-(1-7) (Ang-(1-7) or Asp1-Arg2-VAL3-Tyr4-Ile5-His6-Pro7) was reported in the prevention and treatment of alopecia in cancer patients undergoing chemotherapy. Furthermore, it has been shown in a murine model of alopecia that treatment with Ang-(1-7) induces hair growth and increased the number of mature follicles (WO 01/98325). It is worth mentioning that the route of administration used in patients was subcutaneous. In mice, the Ang-(1-7) was evaluated by the subcutaneous route, as well as a topical formulation of the peptide in carboxymethylcellulose. However, this study did not prove the efficacy of topical formulation, when applied to intact skin. Finally, it a topical formulation of Ang-(1-7) was not claimed in this patent application. The beneficial effect of Ang-(1-7) in alopecia can be attributed to their vasodilation action on blood vessels (Santos et al., 2000). The vasodilation of arterioles present in the dermis improves irrigation of the hair follicles, increasing the supply of nutrients and oxygen. Thus, the cells of the hair follicle increase their proliferation, accelerating hair growth (Santos R A S, Campagnole-Santos M J, Baracho N C V, MAP sources, Silva L C S, Snow L A A, Olivera D R, Caligiome S M, Rodrigues ARV, Gropen Jr. C, Carvalho W S, Silva A C S, Khosla M C Characterization of the new angiotensin antagonist selective goes angiotensin-(1-7): evidence that the actions of angiotensin-(1-7) are mediated by specific angiotensin receptors Brain Res Bull . . . , vol. 35, p. 293-299, 1994). Several analogues of Ang-(1-7) were studied in order to obtain more active peptide (WO 01/98325; Lautner et al. Discovery and characterization of alamandine: a novel component of the renin-angiotensin system Circulation Research Vol . . . 112, p. 1104-1111, 2013). Among these analogues, there is Alamandine, a peptide analog of Ang-(1-7) with replacement of amino acid Asp for Ala in the N-terminal position of Ang-(1-7). This endogenous peptide has vasodilation action similar to that of Ang-(1-7), but binds a different receptor that of Ang-(1-7) (Lautner et al Discovery and characterization of alamandine:. A novel component of the renin-angiotensin system. Circulation Research Vol. 112, p. 1104-1111, 2013). This suggests a possible synergistic action between Ang-(1-7) and alamandine. Despite the extremely promising effect of Ang-(1-7) and some analogues in preventing alopecia (WO 01/98325), the high molecular weight and hydrophilic character of the peptide considerably limit its transdermal absorption and the possibility of obtaining an effective topical formulation. Other inventors have claimed formulations of Ang-(1-7) and analogues, in cyclodextrin, liposomes and biodegradable polymers (U.S. Ser. No. 01/055,097 and WO 03/039434), however, none of these carrier systems are recognized as effective in promoting transdermal absorption of the hydrophilic peptide and no evidence was presented in those works regarding the efficiency of these systems in the treatment of alopecia.

The structure of skin is quite complex, composed of three layers: epidermis, dermis and subcutaneous layer. The epidermis is a stratified avascular layer, the outermost layer of which is called the stratum corneum. This consists of thick layers of dead cells, and is considered the main barrier to passage of substances through the skin by limiting the cutaneous and percutaneous absorption of substances. The dermis is a highly vascularized layer containing the sweat glands, sebaceous glands, hair follicles and nails. The subcutaneous layer is composed mainly of fat, forming a thermal and mechanical barrier (Barry, 2001; Bouwstra et al, 2002;. Cevc et al., 1996). Substances can pass through the skin appendages such as hair follicles, sebaceous glands and sweat secretion ducts through (transcellular route) or between (intercellular route) the cells of the stratum corneum (Barry., 2001; Cevc et al, 1996). Most bioactive agents are not absorbed by the skin; it is known that intermediate molecular weight molecules (>300-500 Da), molecules having a too low oil/water partition coefficient and ionic molecules do not readily penetrate the skin (Barry B W. Novel Mechanisms and devices to enable successful transdermal drug delivery. European Journal of Pharmaceutical Sciences, vol. 14, p. 101-114, 2001b; Cevc G, G Blume, Schatzlein A, D Gebauer, Paul A. The skin: a pathway for systemic treatment with patches and lipid-based carriers Advanced agent. Drug Delivery Reviews, vol. 18, p. 349-378, 1996). Some strategies have been used to increase the transport and absorption of substances through the stratum corneum: moisturizing; electrical methods such as electroporation, iontophoresis, and ultrasound; use of chemical absorption-promoting substances such as water, sulfoxides, surfactants, ethanol (Barry, 2001); and the use of drug carrier systems, such as etossomas and ultradeformable vesicles. For some drugs, these vesicles proved able to promote dermal or transdermal drug absorption, thus facilitating its passage through the skin (WO 91/01596; Cevc G, Blume G. Lipid vesicles penetrate into intact skin owing to the transdermal osmotic gradient and hydration force Biochimica et Biophysica Acta, vol. 1104, p. 226-232, 1992; Touitou And Dayan N, Bergelson L, B Godin, Eliaz M. Ethosomes—novel vesicular carriers for enhanced delivery: characterization and skin penetration properties. Journal of Controlled Release, vol. 65, p. 403-418, 2000).

The ultradeformable vesicles are prepared using specific combinations of phospholipids and surfactants, yielding sufficiently deformable vesicles, which have the ability to cross pores considerably smaller than its own pore size. The authors claim that these vesicles when applied topically under non-occlusive conditions, would cross penetrate the skin spontaneously promoting transdermal absorption of encapsulated drugs (Cevc G, D Gebauer, Stieber J, Schatzlein A, Blume G. Ultraflexible vesicles, Transfersomes, have an extremely low pore penetration resistance and transport therapeutic Amounts for insulin across the intact mammalian skin. Biochimica et Biophysica Acta, vol. 1368, p. 201-215, 1998. Cevc G, Blume G. New, highly efficient formulation of diclofenac for the topical, transdermal administration in ultradeformable drug carriers, Transfersomes. Biochimica et Biophysica Acta, vol. 1514, p. 191-205, 2001). However, there is controversy in the prior art whether this system could be applied to any drug (Elsayed et al, 2007;. El Maghraby et al, 2008;. Benson, 2009). A recent study using calcein as an intermediate molecular weight hydrophilic drug model shows that ultradeformable vesicles promote a reduction of transdermal passage of the hydrophilic drug in vitro and in vivo and do not act as carriers of the drug through the stratum corneum (Bahia et al., 2010). Therefore, the effectiveness of ultradeformable vesicles appears to depend on the particular physicochemical properties of the drug and its compatibility with the composition of the ultradeformable lipid vesicles. Thus, in the case of a hydrophilic peptide such as Ang-(1-7), one can not anticipate a priori the effect of ultradeformable vesicles. (Elsayed M M A Abdallah O R, Naggar V F Khalafallah N M Lipid vesicles for skin delivery of drugs: reviewing three decades of research International Journal of Pharmaceutics, vol. 332, p. 1-16, 2007. El Maghraby G M Barry B W . . . , Williams A C Liposomes and skin: from drug delivery to model membranes European Journal of Pharmaceutical Sciences, vol. 34, p. 203-222, 2008. Benson, H A, 2009. Elastic liposomes for topical and transdermal drug delivery, Curr. Drug Deliv. 6, 217-226).

Another potential advantage of vesicular systems is their ability to accumulate in the hair follicle, which could increase the concentration of the active principle specifically at this location (Li and Hoffman, 1997). This property also can contribute to enhancement of the action of the active principle encapsulated in the prevention or treatment of alopecia (Li, L., Hoffman, R M, 1997. Topical liposome delivery of molecules to hair follicles in mice. J. Dematol. Sci. 14, 101-108). It is noteworthy that the present invention demonstrates for the first time the Ang-(1-7) and its antagonist, D-[Ala7]-Ang-(1-7) or A779 (Santos et al., 1994) and the analogue alamandine have compatibility with ultradeformable vesicles, indicating that its analogues with changes in amino acids at C-terminal and N-terminal positions (of formula Y1-Arg2-VAL3-Tyr4-Ile5-His6-X7 represented by SEQ ID NO: 2) also show compatibility (Santos R A S, Campagnole-Santos M J, Baracho N C V, MAP sources, Silva L C S, Snow L A A, Olivera D R, Caligiome S M, Rodrigues A R V, Gropen Jr. C, Carvalho W S, Silva A C S, Khosla M C. Characterization of the new angiotensin antagonist selective goes angiotensin-(1-7): Evidence that the actions of angiotensin-(1-7) is mediated by specific angiotensin receptors Brain Res Bull, vol 35, p. 293-299, . . . 1994).

Thus, as was found in the prior art, it is concluded that: existing treatments have variable effectiveness among individuals; not all individuals respond the same way, and moreover, interruption of treatment leads to the return of hair loss. Other limitations frequently reported in these treatments are the onset of chronic irritation after application of minoxidil or lotions of a-estradiol and decreased libido in the case of finasteride.

Despite the potential of Ang-(1-7) for the treatment/prevention of alopecia already having been established, their effectiveness has been clearly demonstrated only when administered subcutaneously. Therefore, no prior art topical formulation of Ang-(1-7) is found in the prior art with proven efficacy in inducing hair growth and with potential for treatment and prevention of alopecia.

As an example, there is the hirsutism. Hirsutism is the presence of an excess of abnormal hair growth in women in areas that are common to the growth in males only (Rittmaster, R. Medical treatment of androgen-dependent hirsutism. J. Clin. Endocrinol. Metab., Vol. 80, pp. 2559-2563, 1995). Hirsutism affects 5% to 10% of women depending on the age, menopause and ethnicity, however, in all cases, the presence of hirsutism is very painful for women and subsequently can have a negative impact on their psychosocial life (Cosma, M.; Swiglo, B A; Flynn, D N; Kurtz, M D; LaBelle, M L; Mullan, R J; Erwin, P J, Montori, V M Clinical review: Insulin sensitizers for the treatment of hirsutism: a systematic review and metaanalyses of randomized controlled trials J. Clin Endocrinol. Metab, 2008, 93, 1135-1142.; Sonino, N.; Fava, G A; . . . Mani, E.; Belluardo, P.; Boscaro, M. Quality of life of hirsute women. Postgrad. Med., 1993, 69, 186-189).

Furthermore, over the years, hair is now considered superfluous. The Egyptians were the first to use sandalwood paste, clay and beeswax, ingredients that would be used for waxing, which is still used today. Over the years, the culture of shaving has evolved: earlier only on the legs, then shaving armpits became the major achievement, and during the last two decades of the twentieth century shaving began to be used in the legs, underarms, arms and sometimes in the pubic area. It is observed, therefore, that, throughout history, women have removed hair to make them more attractive. Now, men are also adhering to this fashion for the sake of hygiene and ease (Available in: http://www.cpt.com.br/cursos-estetica-e-beleza/artigos/depilacao-mercado-crescente-tecnicas-produtos-especificos. Accessed on Nov. 21, 2013).

Thus, new depilatory methods now have emerged to get rid of the normal growth in certain regions of the body using aggressive methods of hair removal with waxes, laser and others, which are time-consuming, must be used frequently and also generate high costs. Aimed at solving the problem of hirsutism in women and also to free women and men of the need to resort to hair removal, a formulation containing the receptor antagonist Mas A-779 was developed, which is innovative for the market since a prior art formulation containing receptor antagonist Mas for inhibiting hair growth is not found.

Thus, in the present invention, topical formulations have been developed with proven efficacy in controlling growth of hair and potentially for the treatment and prevention of alopecia; and a formulation with efficacy in inhibiting hair growth. The formulations for the treatment and prevention of alopecia comprising Ang-(1-7) and/or its analogues and the formulation for inhibiting hair growth comprising receptor antagonist Mas, A779 and/or their analogues, are encapsulated in ultradeformable vesicles prepared by specific combinations of phospholipids and surfactants. One can point out the following advantages of these technologies: i) the proposed formulations are safe, because the active ingredient and excipients are chemically defined, with no reports of side effects; ii) the formulations ensure adhesion to the skin and penetration of the active, but with low systemic absorption and high direction of the asset to the hair follicle.

### BRIEF DESCRIPTION OF THE DRAWINGS

FIG. 1. FIG. 1 shows the average length of hair in Swiss mice after topical application of the Ang-(1-7) formulation, the A-779 formulation and vehicle (consisting of the vesicle suspension without peptide). It was applied 50 uL/dose/animal, with 4 doses at intervals of 2 days. *P<0.05 comparing the group receiving formulation containing Ang-(1-7) or A-779 with the group that received the vehicle. One-Way ANOVA, followed by post-test Dunnett.

FIG. 2. Efficacy of topical treatment with peptide formulations of Ang-(1-7) and alamandine in a model of alopecia, C57BL/6 mice undergoing chemotherapy. The data represent the hair growth of mice with induced alopecia after treatment with cyclophosphamide, with or without subsequent topical treatment. The following experimental groups (n=4-6) were: CONTROL, animals that received no treatment; Cyclophosphamide, animals that received cyclophosphamide (150 mg/kg) but without further treatment; CYCLE+V_VAZIO, animals that received cyclophosphamide and then the topical vehicle for 30 days; CYCLE V_ANG+(1-7) animals that received cyclophosphamide and then the topical formulation of Ang-(1-7) for 30 days (10 mg/kg/day); CYCLE+V_ALAMANDINE, animals that received cyclophosphamide and then the topical formulation of Alamandine for 30 days (10 mg/kg/day). **P<0.01 comparing the group “CYCLOPHOSPHAMIDE” with groups CYCLE+V_ALAMANDINA and CYCLE+V_ANG (1-7), Kruskal-Wallis test followed by post-test Dunn's.

FIG. 3. Efficacy of topical treatment with peptide formulations of Ang-(1-7) in the follicular dystrophy in C57BL/6 mice undergoing chemotherapy. The data represent the size of hair follicles in mice with induced alopecia after treatment with cyclophosphamide, with or without subsequent topical treatment. The following experimental groups (n=4-6) were: cyclophosphamide, animals that received cyclophosphamide (150 mg/kg) but without further treatment; CYCLE+V_VAZIO, animals that received cyclophosphamide and then, the topical vehicle for 30 days; CYCLE V_ANG+(1-7) animals that received cyclophosphamide and then the topical formulation of Ang-(1-7) for 30 days (10 mg/kg/day). *P<0.05 Test One-Way ANOVA followed by post-test Dunnett's.

### DETAILED DESCRIPTION

This technology consists of a topical formulation comprising Ang-(1-7) (SEQ ID NO: 1) or analogues thereof and their use in the treatment and prevention of alopecia. The formulation consists of an aqueous suspension of Ang-(1-7) or analogues thereof in the presence of lipid vesicles comprising phospholipid and surfactant. The same can be prepared in a simple manner by injecting an ethanolic solution of phospholipid into an aqueous solution containing the surfactant and Ang-(1-7) or its analogues selected from the group of formula Y1-Arg2-Tyr4-Ile5-VAL3 -His6-X7 (SEQ ID NO: 2). The technology also consists of a topical formulation comprising the Ang-(1-7) receptor antagonist, A-779 (SEQ ID NO: 4) and/or their analogues and their use in inhibiting hair growth.

The surfactant can be ionic or non-ionic; may be a fatty acid or long chain alcohol, alkyl tri-salt/di/methyl-ammonium or alkyl sulfate, monovalent salt of cholate, deoxycholate, glycocholate, glycodeoxycholate, taurodeoxycholate or taurocholate, acyl-or alkanoyl-one dimetilaminoxydo as dodecyl dimethylaminoxydo an acyl- or alkanoyl-N-methylglucamide, N-alkyl-N, N-dimethylglycine, 3-(acyldimethylammonium)-alkanesulfonate, acyl-N-sulfobetaine, a polyethylene-glycol-octylphenyl ether such as ninth ethylene glycol-octylphenyl ether, a polyethylene-acyl ether such as ninth ethylene-dodecyl ether, a polyethylene glycol ether such as isoacil ethylene glycol octahydro-isotridecyl ether, a polyethylene-acyl ether such as octaethylenedodecyl ether, a polyethylene-acyl sorbitan ester such as polyethylene glycol 20 monolaurate (Tween 20) and polyethyleneglycol-sorbitan-monooleate (Tween 80), a poly hydroxyethylene-acyl ether such as poly hydroxyethylene-lauryl, -myristoyl, -cetyl, -stearyl or -oleyl ether as the poly hydroxyethylene-4, -6, -8, -10 or -12, etc. lauryl ether (Brij series) or a corresponding ester such as poly hydroxyethylene-8-stearate (Myrj 45), -laurate or oleate type, or in polyethoxylated castor oil 40 (Cremophor EL), a sorbitan monoalkylated (e.g. in Arlacel or Span) such as sorbitan monolaurate (Arlacel 20, Span 20), sodium oleate, sodium taurate, a fatty acid salt such as sodium elaisate, sodium linoleate or sodium taurate, a lysophospholipid such as n-octadecylene (=oleyl)-glycerophosphatidics acid, or -phosphorylglycerol-phosphorylserine, n-acyl e.g. lauryl or oleyl glycerophosphatidic acid, -phosphorylglycerol or -phosphorylserine, n-tetradecyl-glycerophosphatidic acid, -phosphorylglycerol or -phosphorylserine a palmitoyloyl, elaidoyl, a corresponding vaccenil-lysophospholipid or corresponding short chain phospholipid, or a surfactant polypeptide.

The phospholipid used can be phosphatidylcholine and/or phosphatidylglycerol. The final concentration of phospholipid can vary from 1 to 20% w/v. Ethanol is used with a final concentration of 0 to 30% v/v. The surfactant is added to the aqueous phase in a concentration ranging from 0.1 to 3% w/v, along with the peptide Ang-(1-7) (SEQ ID NO: 1) and/or its analogues, preferably alamandine (SEQ ID NO: 3) in concentrations ranging from 0.1 to 100 μg/mL, or the receptor antagonist Mas A-779 (SEQ ID NO: 4) at concentrations ranging from 0.1 to 100 μg/mL The mean hydrodynamic diameter of the vesicles determined by dynamic light scattering, typically is in the range of 20-500 nm After this step, the size of the vesicles is optionally calibrated, for example by extrusion through a polycarbonate membrane of 100 or 200 nm pore diameter, as previously proposed (Mayer LD, Hope MJ, Culles PR. Vesicles of variable sizes produced by rapid extrusion procedure. Biochimica et Biophysica Acta, vol. 858, p. 161-168, 1986).

To demonstrate the ability of the formulation to promote peptide absorption into the skin and regulation of hair growth, Swiss mice were shaved on the dorsal region and specific regions were selected for application of different formulations. The vehicle consisted of vesicles formed from phosphatidylcholine and sodium cholate. The treatment was performed with 4 doses applied topically at intervals of 2 days. The different groups received the formulation of Ang-(1-7), the formulation of the Mas receptor antagonist (A779) and the vehicle. Nine days after the beginning of treatment, the fur size of the animal was measured in the region of application of the formulations. The average size of the hair was compared between the different groups by one-way ANOVA. According to the results, the formulation of Ang-(1-7) significantly stimulated hair growth (compared to vehicle), while the A779 formulation significantly inhibited the growth. Also, tests in a model of alopecia in C57BL/6 mice undergoing chemotherapy with cyclophosphamide were also performed. The data showed that animal hair growth after chemotherapy-induced alopecia was significantly higher in groups that received topical formulations of Ang-(1-7) and alamandine (daily application for 30 days) compared to the group that did not receive topical treatment. Thus, it was found that the formulation for promoting the absorption of peptides on the skin and ensured its biological effectiveness, which indicates their great potential in the prevention and treatment of alopecia.

For a better understanding of the technology see the following non-limiting examples:

### EXAMPLE 1

### Preparation and Characterization of Topical Formulation of Angiotensin-(1-7)

The formulation consists of a suspension of lipid vesicles of gauged size consisting of a mixture of phospholipid and surfactant in the presence of peptide. In this example, a formulation was prepared simply by injecting an ethanolic solution of soy phosphatidylcholine (SPC 95%, Avanti Polar Lipids Inc.) into an aqueous solution containing sodium cholate (Sigma Co.), and the peptide Ang-(1-7) (Bachem) or its A779 antagonist (Bachem). The ethanolic solution was injected into the aqueous solution using a syringe attached to a needle, and the resulting suspension was kept under magnetic stirring at room temperature. The aqueous solution was composed of 0.15 M NaCl, 0.02 M HEPES, pH 7.4. The final ethanol concentrations, SPC and sodium cholate peptide were 9% (v/v), 8.8% (w/v), 1.2% (w/v) and 10 ug/ml, respectively.

After formation of the suspension, the size of the vesicles was calibrated by repeated filtrations (5 times) through a polycarbonate membrane with a 0.1 μm pore size, at room temperature with 200 psi pressure using a Extruder (Lipex Biomembranes, Canada) as previously described.

The size distribution of the vesicles in the formulation was characterized by dynamic light scattering (Zetasizer, Malvern, UK). The vesicles had a mean hydrodynamic diameter in the 80 to 100 nm range, with a polydispersity index of less than 0.2, indicating a monodisperse population of vesicles.

### EXAMPLE 2

### Influence of Topical Formulations of Ang-(1-7) and its A779 Antagonist in Hair Growth in Mice

The formulations of Ang-(1-7) and its A779 antagonist, prepared as described in Example 1 were evaluated for their ability to modulate hair growth in mice. The evaluation of the efficacy of the formulations was performed using Swiss mice, males, 8 weeks. There were three groups of 4 animals that received the composition of the Ang-1-7), the formulation of the A-779 or vehicle comprising the suspension of vesicles without peptide. The animals were anesthetized and their backs shaved to remove the hair. The formulations were applied topically in the delimited region (50 uL/dose/animal) and 4 doses at intervals of 2 days.

Nine days after the beginning of treatment, hairs were taken for length measurements with a caliper in the region where there was application of the formulations. The average size was compared between the different groups by one-way ANOVA.

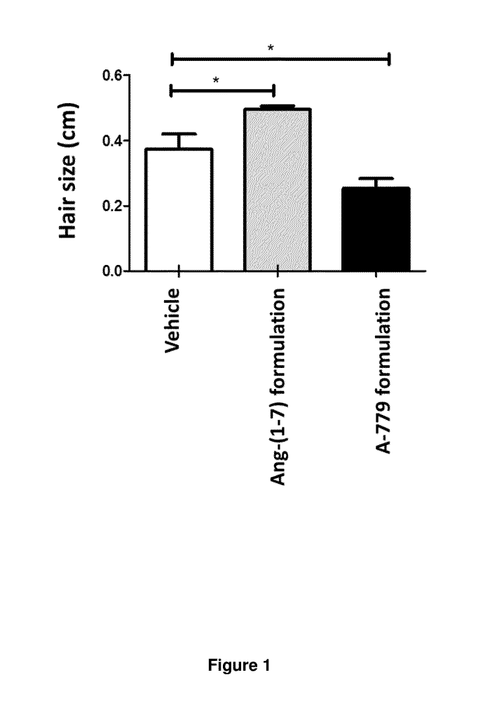

The results, shown in FIG. 1, show that the formulation containing Ang-(1-7) caused a significant increase in hair growth of the animals when compared to vehicle, whereas the formulation containing A779 promoted a reduction in growth when compared to the group treated with the vehicle.

This study demonstrates the effectiveness of topical formulation of Ang-(1-7) in inducing growth of hair in mice. The fact that the formulation A779 inhibits hair growth suggests that the receptor Mas mediates the action of these peptides.

This experiment proves that the formulation promotes the absorption of peptides on the skin and ensures its biological effectiveness, indicating the high potential of the formulation of Ang-(1-7) in the prevention and treatment of alopecia and formulation of growth inhibition in A779 hair.

### EXAMPLE 3

### Efficacy of Topical Formulations of Ang-(1-7) and Alamandine in a Murine Model of Cyclophosphamide-Induced Alopecia

Topical formulations of Ang-(1-7) and alamandine were prepared as described in Example 1 and evaluated for their effectiveness in hair growth induction in C57BL/6 mice following cyclophosphamide induced alopecia (single ip dose of 150 mg/kg) (experimental model described in WO 01/98325).

The following experimental groups (n=4-6) were: CONTROL, animals that received no treatment; Cyclophosphamide, animals that received cyclophosphamide (150 mg/kg) but without further treatment; CYCLE+V_VAZIO, animals that received cyclophosphamide and then the topical vehicle for 30 days; CYCLE V_ANG+(1-7) animals that received cyclophosphamide and then the topical formulation of Ang-(1-7) for 30 days (10 mg/kg/day); CYCLE+V_ALAMANDINE, animals that received cyclophosphamide and then the topical formulation of Alamandine for 30 days (10 mg/kg/day).

After 30 days of treatment, animals were sacrificed; samples of hair and skin were collected from the treated region. The size of the pile yams and the size of the hair follicles of the skin were determined after histological section and stained with H & E.

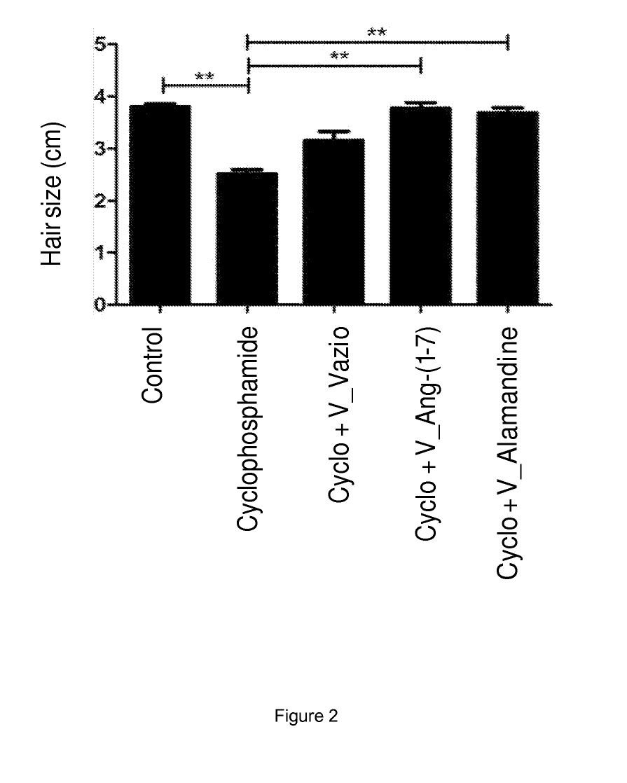

The results for the size of hair, shown in FIG. 2, indicate that treatments with topical formulations of Ang-(1-7) and alamandine promoted a significant increase in the average size of the coat when compared to that of the group treated only with cyclophosphamide (**P<0.01 compared to the group “CYCLOPHOSPHAMIDE” with V_ALAMANDINA CYCLE++V_ANG CYCLE groups and (1-7)).

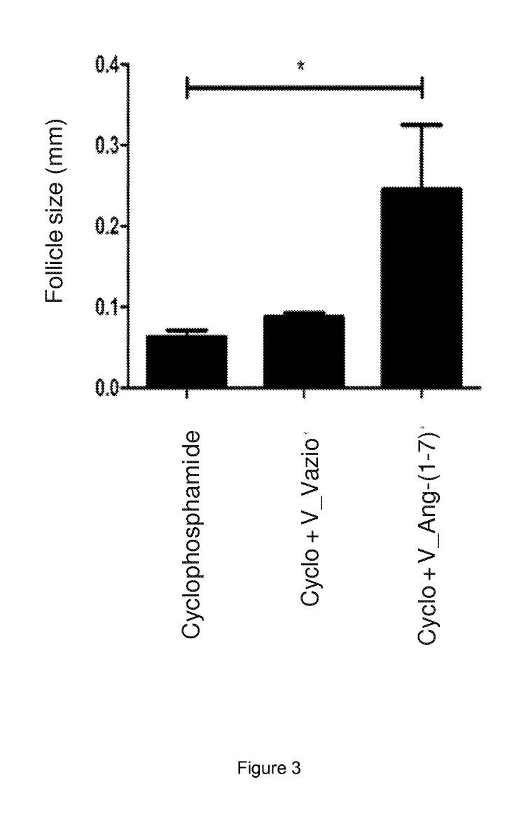

The comparison of the follicle size between the group that received topical treatment with the formulation of Ang-(1-7) and the group treated with Cyclophosphamide is shown in FIG. 3, noting that the topical formulation induced a reversal of Follicular Dystrophy.

Therefore, the set of results demonstrates the effectiveness of topical formulations of Ang-(1-7) and alamandine in a model of alopecia. As these peptides act through different receptors, this study also indicates the potential of association of these two peptides in topical formulation for the prevention and treatment of alopecia.

1. A topical composition comprising 0.1 to 100 μg/ml of one or more angiotensin compounds of SEQ ID NO: 2 wherein X and Y can be any amino acid, and further comprising 0.1 to 3% (w/v) surfactant, 1 to 20% (w/v) phospholipid and 0 to 30% (v/v) ethanol.

2. The topical composition according to claim 1, wherein said angiotensin compounds comprise SEQ ID NO: 1 and alamandine.

3. The topical composition according to claim 1, wherein said angiotensin compounds comprise the alamandine sequence of SEQ ID NO: 3.

4. The topical composition according to claim 1, wherein the surfactant is nonionic or ionic and is selected from the group consisting of a fatty acid; a long chain alcohol; an alkyl salt of trimethyl ammonium, dimethyl ammonium, or methyl ammonium; an alkyl sulfate; a monovalent salt of cholate, deoxycholate, glycocholate, glycodeoxycholate, taurodeoxycholate or taurocholate; an acyl- or alkanoyl-dimethyl amine oxide; an acyl-N-methylglucamide or alkanoyl-N-methylglucamide; N-alkyl-N N-dimethylglycine; 3-(acyldimethylammonium)-alkanesulfonate; N-acyl-sulfobetaine; a polyethylene-glycol-octylphenyl ether; a polyethylene-acyl ether; a polyethylene glycol isoacyl ether; a polyethylene-acyl ether; a polyethylene-acyl sorbitan ester; an acyl polyhydroxyethilene acyl-polyhydroxyethylene ether, or the corresponding ester; polyethoxylated castor oil 40; monoalkylated sorbitan; sodium oleate; sodium taurate; a fatty acid salt; a lysophospholipid; a palmitoloyl; an elaidoyl lysophospholipid or vaccenyl lysophospholipid; an elaidoyl short chain phospholipid or vaccenyl short chain phospholipid and a surfactant polypeptide.

5. The topical composition according to claim 1, wherein the surfactant is a bile salt.

6. The topical composition according to claim 1, wherein the phospholipid is phosphatidylcholine, phosphatidylglycerol, or both.

7. A topical composition comprising 0.1 to 100 μg/mL A-779 (SEQ ID NO: 4) and optionally one or more of its analogues, and further comprising 0.1 to 3% (w/v) surfactant, 1 to 20% (w/v) phospholipid and 0 to 30% (v/v) ethanol.

8. The topical composition according to claim 7, wherein the surfactant is nonionic or ionic and is selected from the group consisting of a fatty acid; a long chain alcohol; an alkyl salt of trimethyl ammonium, dimethyl ammonium, or methyl ammonium; an alkyl sulfate; a monovalent salt of cholate, deoxycholate, glycocholate, glycodeoxycholate, taurodeoxycholate or taurocholate; an acyl- or alkanoyl-dimethyl amine oxide; an acyl-N-methylglucamide or alkanoyl-N-methylglucamide; N-alkyl-N N-dimethylglycine; 3-(acyldimethylammonium)-alkanesulfonate; N-acyl-sulfobetaine; a polyethylene-glycol-octylphenyl ether; a polyethylene-acyl ether; a polyethylene glycol isoacyl ether; a polyethylene-acyl ether; a polyethylene-acyl sorbitan ester; an acyl ether, or the corresponding ester; polyethoxylated castor oil 40; monoalkylated sorbitan; sodium oleate; sodium taurate; a fatty acid salt; a lysophospholipid; a palmitoloyl; an elaidoyl lysophospholipid or vaccenyl lysophospholipid; an elaidoyl short chain phospholipid or vaccenyl short chain phospholipid and a surfactant polypeptide.

9. The topical composition according to claim 8, wherein the surfactant is a bile salt.

10. The topical composition according to claim 7, wherein the phospholipid is phosphatidylcholine, phosphatidylglycerol, or both.
